(19) United States
(12) Patent Application Publication
(10) Pub. No.: US 2023/0382944 A1
(43) Pub. Date: Nov. 30, 2023

(54) NOVEL COCRYSTALS OF DEXAMETHASONE

(71) Applicants: Versitech Limited, Hong Kong (CN); Advanced Biomedical Instrumentation Centre Limited, Hong Kong (CN)

(72) Inventors: Shing Fung CHOW, Hong Kong (CN); Kam Hung LOW, Hong Kong (CN); Si Nga WONG, Hong Kong (CN)

(21) Appl. No.: 18/307,992

(22) Filed: Apr. 27, 2023

Related U.S. Application Data
(60) Provisional application No. 63/365,396, filed on May 26, 2022.

(51) Int. Cl. C07J 9/00; C07C 37/84; A61K 9/00

(52) U.S. Cl. CPC C07J 9/00; C07B 2200/13; A61K 9/0075; C07C 37/84

(57) ABSTRACT

The subject invention pertains to cocrystals of dexamethasone (DEX) and a benzenediol cocrystal composed of a 1:1 molar ratio of DEX and the benzenediol. The benzenediol can be catechol (CAT) or resorcinol (RES). The DEX and benzenediol cocrystal are formed by grinding crystalline DEX where the crystalline benzenediol are combined in the 1:1 molar ratio. Grinding can be performed at room temperature. Cocrystals can be thermally annealed or exposed to humidity to enhance cocrystal formation. The DEX−CAT or DEX-RES cocrystals can be included in a medicament for use in treatments for allergies, asthma, rhinitis, cancer, diabetes, anemia, ulcers, and viral infections. The DEX−benzenediol cocrystal can be sieved to provide particles that are in the range of 10 to 45 μm that can be used for intranasal administration with improved dissolution performance.

### CROSS-REFERENCE TO RELATED APPLICATION

This application claims the benefit of U.S. Provisional Application Ser. No. 63/365,396, filed May 26, 2022, which is hereby incorporated by reference in its entirety including any tables, figures, or drawings.

### BACKGROUND OF THE INVENTION

Recent advances in cocrystallization have sparked tremendous impetus for its diverse applications in modifying physicochemical properties of drugs, not only solubility, dissolution rate and bioavailability, but also manufacturability, luminescence, and organoleptic properties. Cocrystal screening and preparation methods at early development can mainly be divided into two categories: solution-based crystallization and solid-state crystallization. The former confers advantages over the other in the pharmaceutical industry thanks to the ease of scalability and critical attribute control (e.g., size, morphology, and polymorphic form). However, the thermodynamics and kinetics of the cocrystallization process show intriguing interplay in different solvent systems, leading to unpredictable outcomes. The incongruent solubility of the cocrystal formers also augments the tendency of the less soluble former to precipitate from solution before its concentration reaches the labile zone for spontaneous cocrystallization. Solid-state cocrystallization, such as neat grinding, can negate the solvent effect, with the apparent equilibrium achieved in specific conditions often corresponds to the experimental conditions used but not to the thermodynamic equilibrium. Hence, it is a useful alternative for discovery of the hidden metastable cocrystals which is not accessible by solution crystallization.

As a kinetic process, reaction of mechanochemical means is induced by a sufficient energy input which surpasses the relative strength of the supramolecular interactions responsible for assembling the crystalline substrates together. The molecular diffusion process involved in cocrystallization via mechanochemistry can be mediated by one of three high energy transition intermediates with enhanced molecular mobility, including (i) vapor, (ii) liquid eutectic, and (iii) amorphous phases. For neat grinding, formation of an amorphous intermediate is the most common mechanism, especially when limited mass transfer happens. For instance, a 60-min grinding only results in 60% of the phenazine and mesaconic acid cocrystal, whereas the rest remains as an amorphous mixture. Similarly, co-grinding of carbamazepine and saccharine under cryogenic conditions led to formation of amorphous phase, which subsequently transforms into cocrystal during storage at room temperature. The transformation efficiency is strongly contingent on the annealing temperature and moisture. These findings carry important implications, particularly when a cocrystal screening involves coformers with high glass transition temperature (T_{g}) and glass forming ability (GFA), as there exists a risk of the reactants being entrapped in the undesirable amorphous state and the complete conversion to a stable cocrystal state might take days, months, or even years, depending on the storage conditions.

Dexamethasone (DEX), 9-fluoro-11β,17,21 -trihydroxy-16α-methylpregna-1,4-diene-3,20-dione), a commonly used synthetic glucocorticoid, is indicated for the treatment of a wide range of acute and chronic inflammatory conditions, including allergic states, dermatological, ophthalmic, rheumatic, and neurological diseases, etc. It is recognized as one of the medicines able to reduce mortality in patients infected with COVID-19 who are critically or severely ill, with a recommendation by the World Health Organization (WHO). However, DEX is poorly soluble in water with an aqueous solubility of around 90 mg/L at 25° C. and exhibits a poor dissolution performance. No DEX cocrystal is known. Identification of complementary cocrystal formers is typically deemed as a rate limiting step to successful cocrystal development. The study of supramolecular chemistry reveals that most active pharmaceutical ingredients present polar groups rich in predictable interactions conducive to cocrystal formation, primarily R₂² (8) hydrogen-bonding synthons. DEX features hydroxyl groups and is not a solitary moiety conferring few predictable interactions that are sufficiently strong or versatile to favor cocrystallization.

### BRIEF SUMMARY OF THE INVENTION

Embodiments are directed to a dexamethasone (DEX) and benzenediol cocrystal that is composed of a 1:1 molar ratio of DEX to a benzenediol. The benzenediol can be catechol (CAT) or resorcinol (RES). The DEX and benzenediol cocrystal can have a particle size below 63 μm, for example, 10-45 μm.

Another embodiment is directed to a method of preparing the DEX and benzenediol cocrystal, where crystalline DEX and a crystalline benzenediol are combined in a 1:1 molar ratio to form a DEX+benzenediol mixture that is subsequently ground to form DEX−benzenediol cocrystals. The mixing and grinding can be performed at room temperature. The resulting DEX−benzenediol cocrystals can be thermally annealed and/or exposed to humidity. The DEX−benzenediol cocrystals can be sieved. The sieved DEX−benzenediol cocrystal can have particles that are below 63 μm, for example, 10-45 μm.

Other embodiments are directed to a medicament that includes the DEX−benzenediol cocrystal. The medicament can be used in treatments for allergies, asthma, rhinitis, cancer, diabetes, anemia, ulcers, and viral infections. The medicament can be used with intranasal administration.

### BRIEF DESCRIPTION OF THE DRAWINGS

FIG. 1 shows chemical structures of DEX and benzene diol and triol coformers for cocrystals, according to embodiments.

FIG. 2A shows a temperature—composition phase diagram of DEX−CAT cocrystal system, indicating the 1:1 stoichiometry.

FIG. 2B shows a temperature—composition phase diagram of DEX−RES cocrystal system, indicating the 1:1 stoichiometry.

FIG. 3 shows PXRD patterns of the 1:1 physical mixture formed between DEX and hydroquinone (HYQ), hydroxyquinol (HXQ), pyrogallol (PYR), and phloroglucinol (PHL) by neat grinding and the constituents therein.

FIG. 4 shows PXRD patterns of the 1:1 DEX−benzenediol cocrystal systems using catechol (CAT) and resorcinol (RES), according to embodiments of the invention, obtained from neat grinding after annealing, and Rietveld—refined simulated patterns.

FIG. 5 shows DEX−benzenediol cocrystal systems, CAT and RES, according to embodiments, by PXRD after one-month storage at 25° C./75% RH.

FIG. 6 shows DSC profiles of the DEX−benzenediol cocrystal systems, according to embodiments, after annealing relative to their starting materials.

FIG. 7 shows combined TGA plots of the DEX−benzenediol cocrystal systems, according to embodiments, compared with the starting materials.

FIG. 8A shows DSC profiles of DEX−CAT, according to an embodiment, at various grinding periods.

FIG. 8B shows DSC profiles of DEX−RES, according to an embodiment, at various grinding periods.

FIG. 9A show thermal analysis of the quench cooled DEX−CAT via DSC heating/cooling/heating plots that demonstrate transformation efficiency from coamorphous to cocrystal phase at different annealing temperatures.

FIG. 9B show thermal analysis of the quench cooled DEX−RES via DSC heating/cooling/heating plots that demonstrate transformation efficiency from coamorphous to cocrystal phase at different annealing temperatures.

FIG. 10 shows least-squares overlay (RMS=0.0845) for six pairs of quaternary carbon atoms of DEX) of the structures of DEX−CAT (dark) and DEX−RES (light) with an additional pair of DEX (−x, ½+y, 1−z) and CAT (−x, −½+y, 1−z) molecules was generated for a better comparison with the asymmetric unit of DEX−RES.

FIG. 11 shows crystal structure of the 1×1×2 unit cell of the DEX−CAT cocrystal system view along the b-axis without hydrogen atoms and unit cell axes for clarity indicating intermolecular hydrogen bonding between (DEX−CAT), (DEX−DEX), and (CAT−CAT).

FIG. 12 Crystal structure of the 1×1×1 unit cell of the DEX−RES cocrystal system view along the b-axis without hydrogen atoms and unit cell axes for clarity indicating intermolecular hydrogen bonding between (DEX−RES), (DEX−DEX), and (RES−RES).

FIG. 13 shows FTIR spectra of DEX−benzenediol cocrystals after annealing and of the starting materials.

FIG. 14A shows an SEM image of sifted DEX at 5000× magnification.

FIG. 14B shows an SEM image of sifted DEX−CAT at 5000× magnification.

FIG. 14C shows an SEM image of sifted DEX−RES at 5000× magnification.

FIG. 15 shows a composite plot indicating dissolution profiles for DEX, DEX cocrystals (DEX−CAT, DEX−RES), and physical mixture of DEX and benzenediols (DEX+CAT, DEX+RES) with particle sizes below 63 μm in pH 5.5 simulated nasal fluid (n=3).

FIG. 16 shows a bar chart of NGI dispersion data of spray dried DEX−RES formulations (n=3) where S1-S7 presents impactor stages 1-7 with upper aerodynamic cutoff diameter in parentheses and MOC is the micro-orifice collector in the NGI.

### DETAILED DISCLOSURE OF THE INVENTION

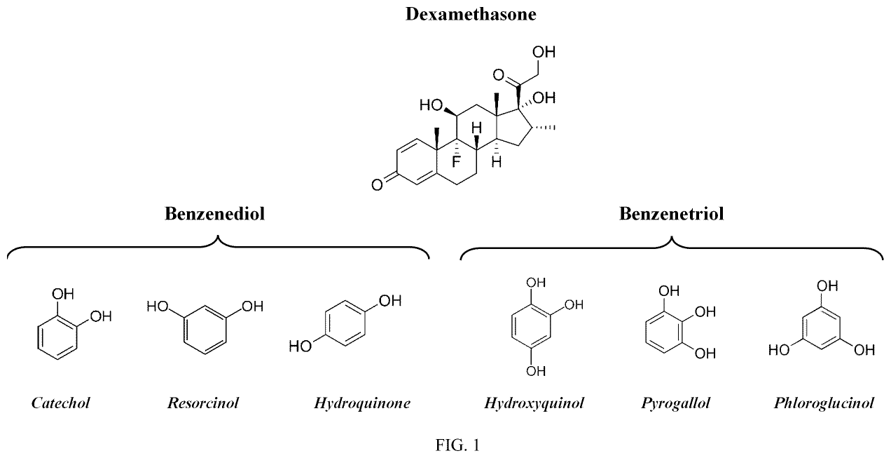

In embodiments of the invention cocrystals comprising Dexamethasone (DEX) are formed by mechanochemical grinding. The DEX cocrystals display improved pharmaceutical properties through OH . . . OH heterosynthons, that are less energetically preferred to other heterosynthons. The DEX cocrystals were identified upon examination of structurally similar polyphenolics differing in the positions and numbers of hydroxyl groups on the benzene ring. Examined coformers, including catechol (CAT), resorcinol (RES), hydroquinone (HYQ), hydroxyquinol (HXQ), phloroglucinol (PHL), and pyrogallol (PYR), as shown in FIG. 1. Although not all coformers are on the GRAS list, many of them are reported to possess antioxidant and anti-inflammatory effects that are potentially synergistic with DEX by means of cocrystal formation. In one embodiment, RES derivatives exhibit a wide variety of pharmaceutical uses, including treatment for inflammation conditions (e.g., allergy, asthma, and rhinitis), cancer, diabetes, anemia, ulcers, and viral infections, etc. RES exerted anti-allergic effects through blockage of histamine release. Analysis of the unique supramolecular interactions being engaged suggest a mode for designing DEX cocrystals with other phenolic compounds, including herbal medicines that are polyphenol and flavonoid exhibiting similar chemical structures with the model coformers.

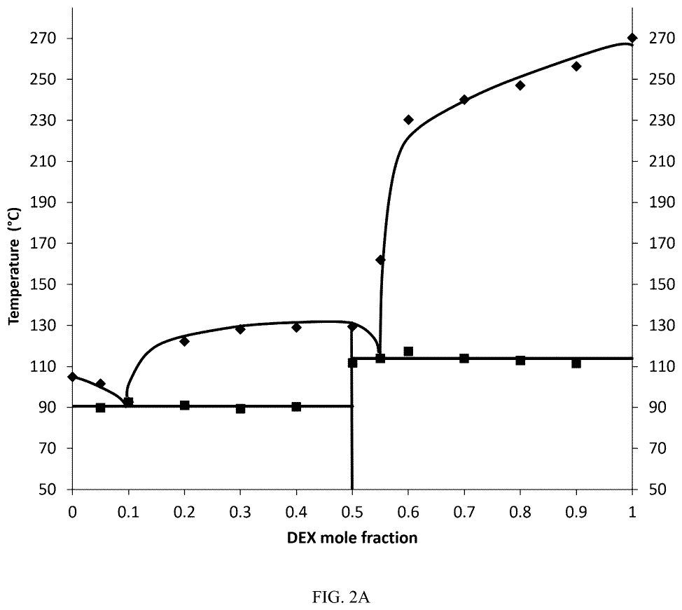

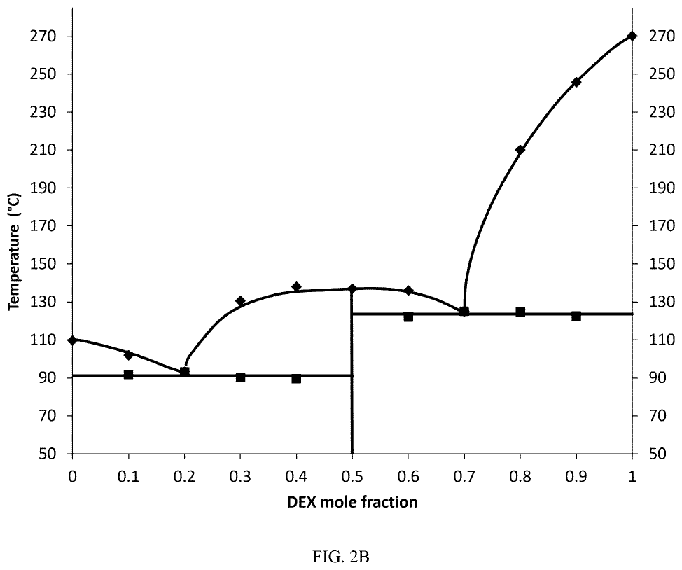

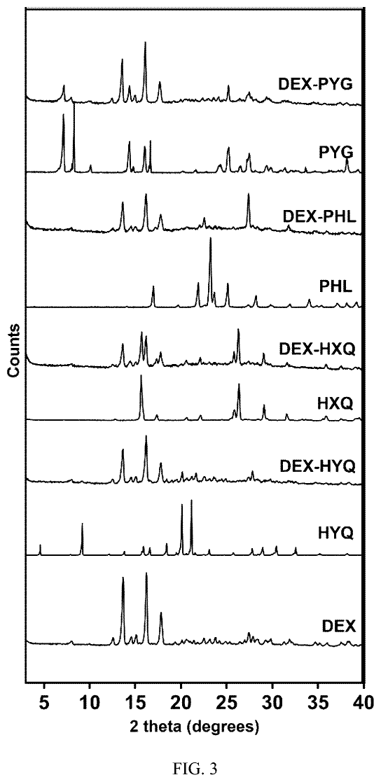

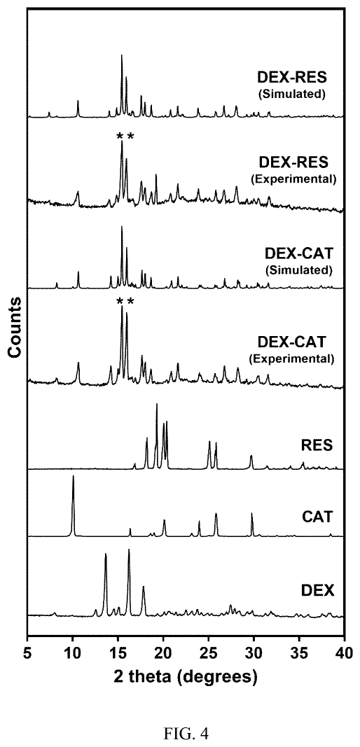

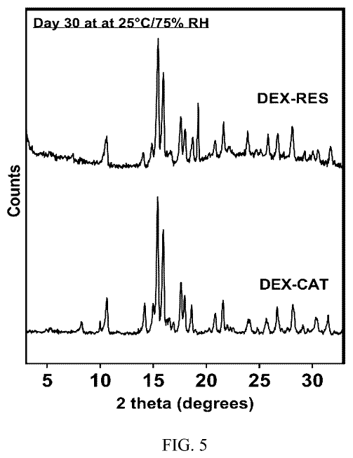

In embodiments, the cocrystal includes one or more coformers of the structure:

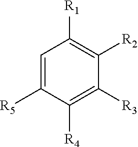

where independently R₁, R₂, R₃, R₄, and R₅ are H, OH, CO₂H, NH₂, or SH and wherein at least two of R₁, R₂, R₃, R₄, and R₅ are not H. The cocrystal forms with an m:n molar ratio of DEX to the coformer, where m is 1, 2, 3, or 4, and n is 0.5, 1, 2, 3, or 4, for example, where m is 1 and n is 0.5, 1, or 2. The coformer can be selected from benzenediols, benzenetriols, benzenetetrols, and pentahydroxybenzene, such as, but not limited to, catechol or resorcinol. Of benzenediol and benzenetriol coformers, phase pure DEX−CAT and DEX−RES cocrystals form in a 1:1 stoichiometric ratio when obtained by mechanochemical grinding, although not by other tested methods, including solvent evaporation and melt crystallization, as in indicated in FIGS. 2A and 2B. A local maximum melting temperature at 0.5 DEX mole fraction of either cocrystal former can be observed in the temperature-composition diagrams of DEX−CAT and DEX−RES, respectively, confirming their 1:1 stoichiometry. Other of the tested potential coformers only resulted in simple physical mixture as indicated in FIG. 3. Anhydrous DEX cocrystals were obtained as a polycrystalline free-flow powder with micron-scale particle size. As shown in the FIG. 4, the PXRD pattern of the cocrystal samples exhibited a number of unique diffraction peaks (DEX−CAT: 2θ=10.56°, 14.12°, 15.34°, 15.87°, 17.54°, 17.89°, 18.53°, 21.51°, DEX−RES: 2θ=15.36°, 15.85°, 17.50°, 17.91°, 18.61°, 21.51°), while the characteristic peaks corresponding to DEX (2θ=12.56°, 13.64°, 14.57°, 15.10°, 16.22°, 17.84°), CAT (2θ=10.09°, 16.36°, 20.12°), and RES (2θ=16.88°, 18.22°, 19.29°, 20.08°, 20.40°) were absent, in agreement with the simulated PXRD patterns. Notably, the resulting DEX−CAT and DEX−RES cocrystals revealed highly similar PXRD patterns with various shared signature diffraction peaks. For example, an intensive duplet appearing in the range of 2θ at approximately 15.4° and 15.9° was clearly observed, as denoted by * in FIG. 4, followed by another weaker duplet at higher angles, i.e., 2θ=˜17.5° and ˜17.9°. The presence of peaks at similar 2θ in PXRD suggests the isomorphic nature of DEX−CAT and DEX−RES have a closely resembling molecular scaffolds. Regarding the solid-state stability, the cocrystals maintained their physicochemical stability under high humidity condition (25° C./75% RH) for one month as indicated in FIG. 5 and Table 1, below.

TABLE 1
Drug assay before and after storage of the DEX-benzenediol
cocrystal systems under 25° C./75% RH in day 0, 7, and 30.

| | Time | % of DEX | % Difference |
|---|---|---|---|
| Compound | Interval | Remaining (n = 3) | (Day 0-Day 30) |
| DEX | Day 0 | 98.77 ± 2.97 | 4.27 ± 5.14 |
| | Day 7 | 97.23 ± 4.27 | (p = 0.23) |
| | Day 30 | 94.50 ± 3.00 | |
| DEX-CAT | Day 0 | 97.83 ± 2.32 | 2.38 ± 5.49 |
| | Day 7 | 96.73 ± 4.72 | (p = 0.46) |
| | Day 30 | 95.45 ± 3.35 | |
| DEX-RES | Day 0 | 99.65 ± 2.58 | 4.50 ± 4.07 |
| | Day 7 | 97.18 ± 3.87 | (p = 0.19) |
| | Day 30 | 95.15 ± 3.17 | |

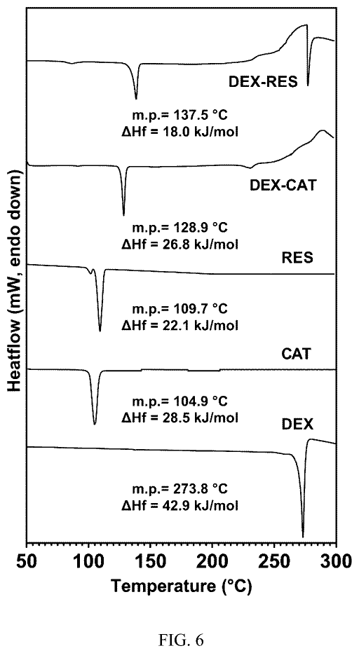

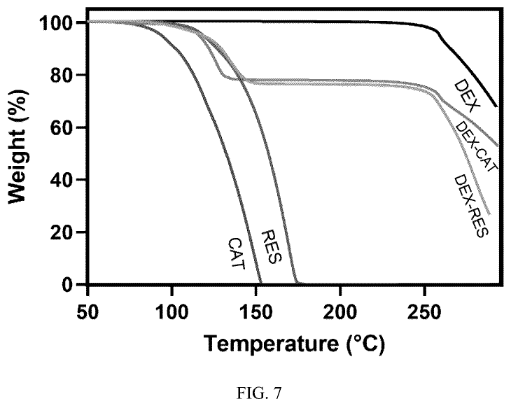

The thermal properties of the cocrystals are given in Table 2, below, where the DEX cocrystals, DEX−RES (133.8° C.) melts at a higher temperature than DEX−CAT (128.4° C.). This may reflect the achievement of a slightly higher packing efficiency by the RES, leading to stronger interactions in DEX−RES in the state of dynamic equilibrium. Both cocrystals show a melting endotherm intermediate to DEX (273.2° C.) and the coformers (CAT: 104.9° C.; RES: 109.3° C.), excluding a possibility of eutectic formation, as indicated in FIG. 6. A non-hygroscopic state of both cocrystals is supported by TGA data, which demonstrates insignificant steps of weight loss prior to melting, as indicated in FIG. 7. Particularly, an unusual depression of crystal lattice strength upon cocrystallization is observed. The molar enthalpy of fusion (ΔH_{f}) of DEX−CAT is ˜60% lower than the sum of those of its individual constituents [ΔH_{f(DEX)}+ΔH_{f(CAT)}], whereas the ΔH_{f} of DEX−RES is ˜70% lower than ΔH_{f(DEX)}+ΔH_{f(RES)}. This contrasts with the vast majority of organic cocrystals, of which the ΔH_{f} lies between or above their constituted counterparts.

TABLE 2
Melting temperature and heat of fusion of DEX, the polyphenolic
coformers, and each DEX cocrystal systems (n = 3).

| Sample | Melting point (° C.) | ΔH_{f} (kJ/mol) |
|---|---|---|
| DEX | 273.8 ± 0.5 | 42.9 ± 1.5 |
| CAT | 104.9 ± 0.3 | 28.5 ± 0.2 |
| RES | 109.7 ± 0.6 | 22.1 ± 0.2 |
| DEX-CAT | 128.9 ± 1.1 | 26.8 ± 0.3 |
| DEX-RES | 137.5 ± 0.8 | 18.0 ± 1.5 |

In embodiments of the invention, the synthesis of metastable cocrystals is achieved by a single production method, mechanochemical grinding. Efforts in cocrystallization via slow evaporation resulted in phase separation into individual constituents, owing to incongruent solubilities of the cocrystal formers in the solvent system. Attempts at rapid evaporation and melt crystallization preferentially formed amorphous mixtures. With its complex structure and having a molecular weight greater than 300 g/mol, it appears that DEX would confer a high glass forming ability that is susceptible to transformation to its amorphous state in kinetic environment, especially when subject to mechanical treatment, i.e., a kinetic process.

When a cocrystal is not obtainable by solvent-mediated methods, solid-state cocrystallization, such as contact formation and grinding, are useful to minimize the complicating effects of solubility and solvent competition. The application of mechanical stress during grinding fractures DEX crystals to promote molecular diffusion through the crystal surfaces. The mechanochemical cocrystallization generates an activation energy sufficient to surmount the low affinity existing between DEX and CAT/RES exceeding the kinetic barrier for cocrystal synthesis. Such a self-assembly process in the solid phase is stabilized by a weak hydrogen bonding network.

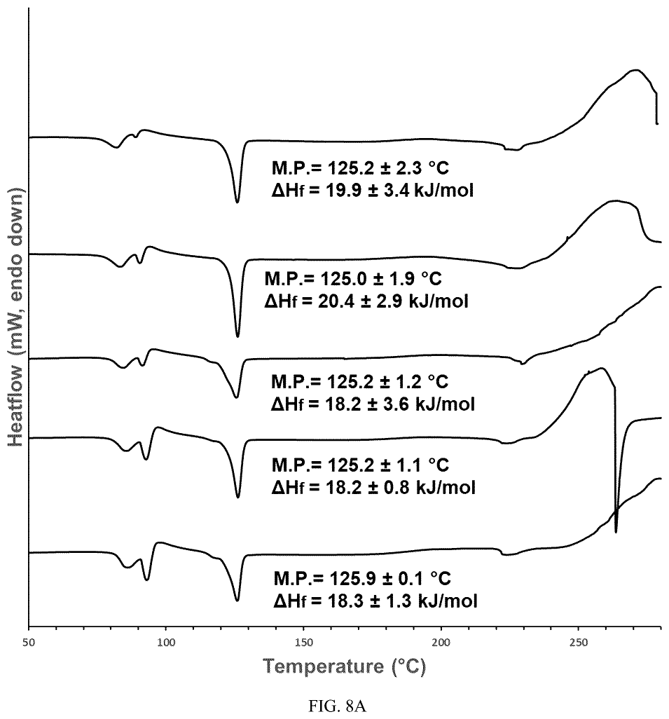

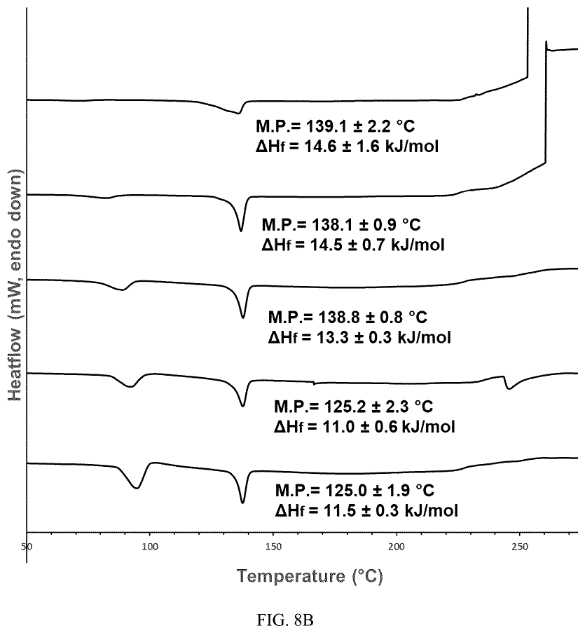

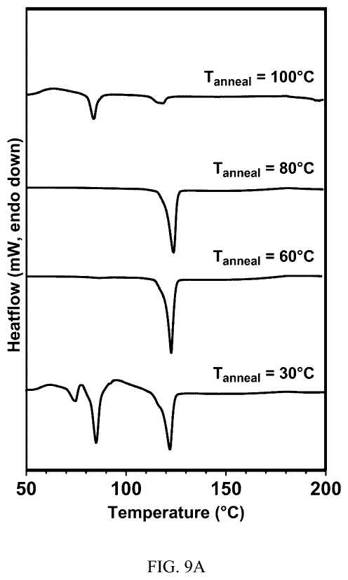

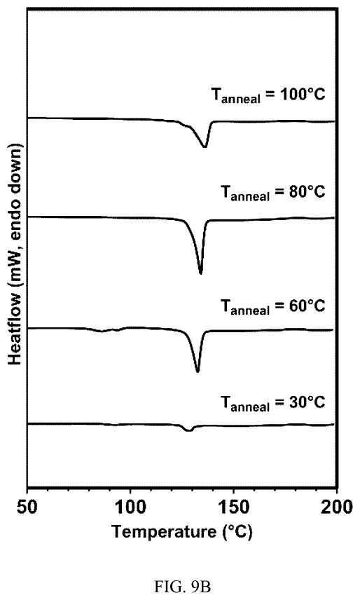

The effect of grinding time on the crystal lattice strength was studied as indicated in FIGS. 8A and 8B. The DSC curves of DEX cocrystals obtained immediately after a 3 min grinding exhibits glass transition temperatures (T_{g}) at around 75° C., followed by recrystallization and melting of the recrystallized cocrystals, which were consistently observed upon further grinding to 10, 20 and 30 min, such that an equilibrium structure appears to be reached within 3 min. Hence, cogrinding the binary mixture at room temperature, which is far below the T_{g}, kinetically entraps DEX and the benzenediol molecules in a partially coamorphous state with high instability, which then transformed to cocrystal during storage in a moisture-facilitated manner. Cocrystal formation during storage after mechanical activation is known to be mediated by an amorphous phase depending on the annealing conditions (i.e., temperature and humidity). DEX cocrystals evaluated from their undercooled melt at different annealing temperatures (30, 60, 80, and 100° C.) during a DSC heat-cool-heat cycle show that energy input during thermal annealing at 60° C. and 80° C. drove the disorderly molecular orientation to complete recrystallization of DEX−CAT to produce phase pure DEX cocrystal with the highest crystallinity and ΔH_{f}(kJ/mol), as shown in FIG. 9A. A similar trend is observed in DEX−RES on the basis of structural resemblance, as shown in FIG. 9B. Annealing temperatures that deviate from the optimal value have inhibitory effect on phase transformation. Short-range disordered coamorphous solid in high-energy states are formed when annealing occurs near room temperature (30° C.), below the T_{g}'s of the samples. The re-cocrystallization process is resisted since the materials are more vicious with lower molecular mobility in the brittle glassy state, compared with its supercooled liquid state counterpart. This resulted in the observation of several melting endotherms in the DSC profile of DEX−CAT, while an intact peak is almost undetected for DEX−RES suggesting the coamorphous phase is dominate. A high annealing temperature (100° C.), approaching the onset of melting of the DEX cocrystals, led to similar outcomes. To accelerate the phase transformation at room temperature, the ground samples were equilibrated at a 75% relative humidity using saturated sodium chloride solution in desiccators. Phase transformation of a coamorphous system into a cocrystal can result upon storage. It is postulated that the hygroscopic coamorphous state undergoes successive stages in moisture-facilitated cocrystallization, namely water adsorption from the atmosphere, followed by local reorganization of the adsorbate into a thermodynamically stable geometry. This is attainable due to the potent plasticizing effect of water, which increases the cocrystallization rate by lowering the T_{g} and elevating molecular mobility and complementarity in the solid state. Successful transformation to cocrystal is only possible under an optimal annealing condition, for example, elevated temperature and humidity that usually does not match the normal storage condition.

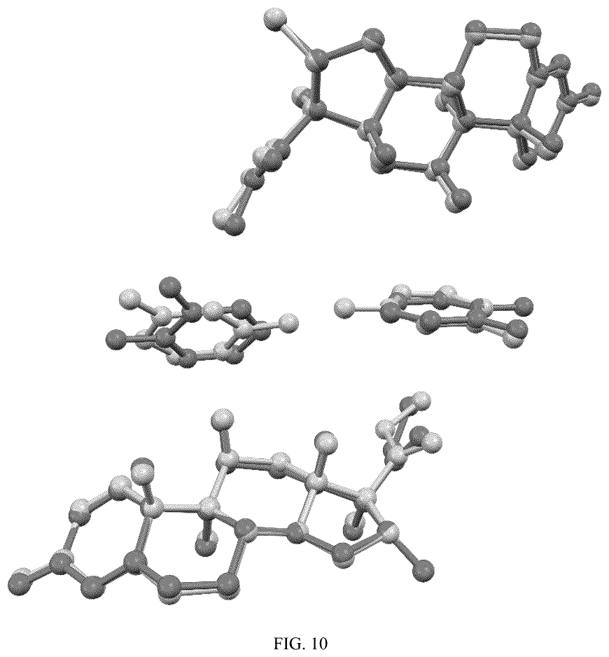

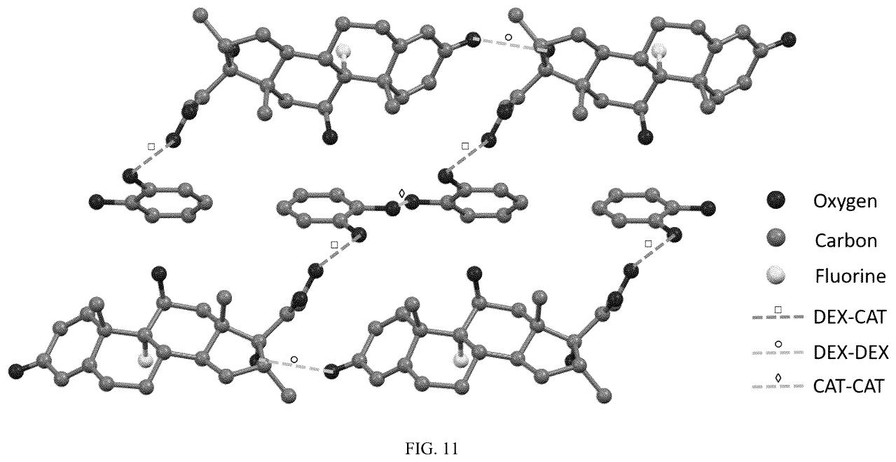

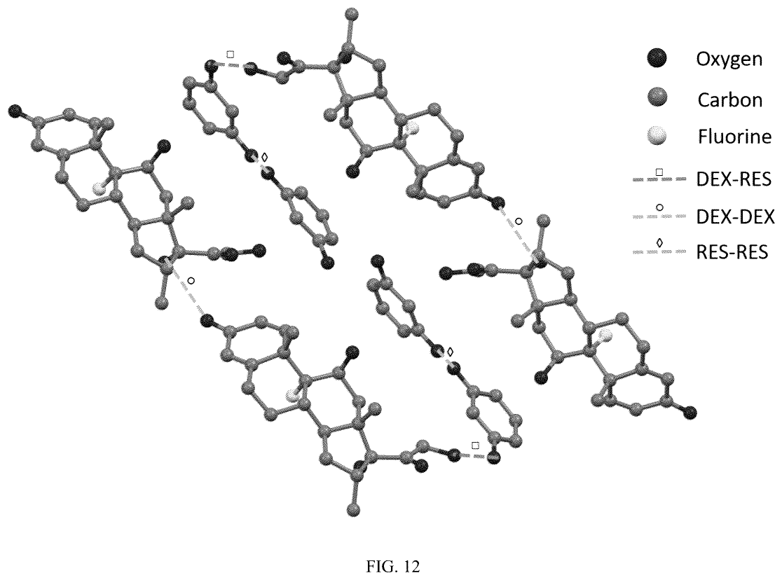

The three-dimensional molecular structures and spatial arrangements of the two DEX are shown in the Rietveld refinement of X-ray powder diffraction patterns. Crystals prepared from neat grinding were submicron-sized and weakly diffracting, which are not suitable to single-crystal X-ray diffraction analysis. The crystal structures obtained from the Rietveld refinement of their corresponding PXRD data confirmed that DEX−CAT and DEX−RES adopt similar crystal packing, as shown in FIG. 10, where the crystal packing driving force is dominant by intermolecular hydrogen bonding. DEX−CAT and DEX−RES crystallize in a non-centrosymmetric monoclinic P2₁ space group. The asymmetric unit of DEX−CAT comprises one molecule of DEX and one molecule of CAT, whereas that of DEX−RES comprises two pairs of crystallographically independent DEX and RES molecules. In the homogeneous DEX crystal structure, the compound crystallizes in the orthorhombic P2₁2₁2₁ space group, having intramolecular hydrogen bonding in the alpha-hydroxy ketone group and intermolecular hydrogen bonding between the tertiary hydroxyl group and the cyclic ketone oxygen of the adjacent molecule, as well as between the secondary hydroxyl group and the alcohol oxygen of the alpha-ketol of the adjacent molecule. Besides the homo-intermolecular hydrogen bonding between the tertiary hydroxyl group and the cyclic ketone oxygen in the DEX molecules, the DEX−CAT and the DEX−RES crystal lattices are stabilized by the O—H . . . O hydrogen bonding between the O5 atom on the hydroxyl group of the alpha-ketol group in DEX as an acceptor and the phenolic hydroxyl group of the CAT or RES as a donor, forming an OH . . . OH heterosynthon with a hydrogen bond length of 2.50(3) Å and 2.51(5) Å, respectively, as illustrated in FIGS. 11 and 12. It is observed that both the CAT or RES molecules form homo- and hetero-intermolecular hydrogen bonding simultaneously, which makes results in a unique hydrogen-bonded interlayer sandwiched between the DEX molecules, interrupting the intermolecular interactions in the original DEX packing, which may allow different pharmaceutical properties (e.g., solubility and dissolution rate) relative to the parent DEX. Selected crystallographic data and structure refinement results are shown in Table 3, below.

TABLE 3
Selected crystallographic data and structure refinement results of
DEX-CAT and DEX-RES.

| | DEX-CAT | DEX-RES |
|---|---|---|
| Moiety formula | C₂₂H₂₉FO₅, C₆H₆O₂ | C₂₂H₂₉FO₅, C₆H₆O₂ |
| Formula weight | 502.576 | 502.576 |
| Crystal system | Monoclinic | Monoclinic |
| Space group | P2₁ | P2₁ |
| Temperature/K | 293 | 293 |
| Appearance | White powder | White powder |
| a/Å | 16.9895(7) | 18.9012(7) |
| b/Å | 6.1074(2) | 6.10485(19) |
| c/Å | 12.0498(9) | 22.6364(15) |
| α/° | 90 | 90 |
| β/° | 101.833(6) | 109.250(4) |
| γ/° | 90 | 90 |
| Volume/Å³ | 1223.73(11) | 2465.9(2) |
| Z | 2 | 4 |
| ρ_{calc}/gcm³ | 1.364 | 1.354 |
| 2θₘᵢₙ, 2θₘₐₓ/° | 3.00, 59.960 | 3.00, 69.94 |
| 2θₛₜₑₚ/° | 0.02 | 0.02 |
| Number of reflections | 410 | 1239 |
| Final R_{wp}/Rₑₓₚ/R_{I} | 0.076/0.034/0.092 | 0.087/0.035/0.093 |

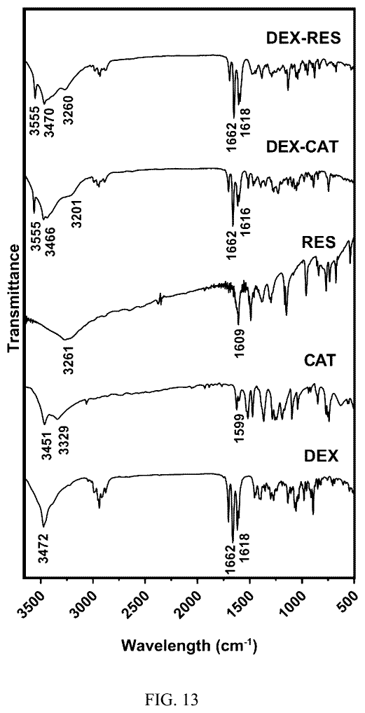

FTIR spectra in FIG. 13 and Table 4, below, displays O—H stretching vibrations that reflect an alteration of the molecular environment upon cocrystallization. DEX−CAT and DEX−RES exhibited new broad absorption peaks between 3200 and 3700 cm⁻¹, which are assigned to phenolic O—H stretching (DEX−CAT: 3466, 3201 cm⁻¹; DEX−RES: 3470, 3260 cm⁻¹). The decrease in wavelength of phenolic O—H stretching from 3472 cm⁻¹ (DEX) to a lower frequency implies involvement of the O—H group in an intermolecular hydrogen bond network without proton transfer. For raw CAT, an intramolecular O—H . . . O hydrogen bond in a six-member ring is formed between the two hydroxyl groups, one acting as the hydrogen donor or bonding group (OH_{b}), and the other acting as the acceptor or free group (OH_{f}). The intramolecular hydrogen bond is relatively weak compared to intermolecular hydrogen bonds. Upon cocrystallization via mechanochemistry, this bond is broken to form an “open” structure, CAT in DEX cocrystal is present exclusively in homodimers where OH_{b} is bonded with another CAT molecule and OH_{f} donates a hydrogen atom to form an intermolecular hydrogen bond with the oxygen atom O5 in the hydroxyl group of DEX. This is reflected by the additional shoulder appearing at 3555 cm⁻¹, which is indiscernible in the starting materials, and the new broad band appearing at lower frequency, i.e., 3201 cm⁻¹. DEX−RES exhibits similar vibrational characteristics as DEX−CAT. Intramolecular hydrogen bonding of CAT is strengthened over interacting with bases, such as DEX, in inert solvents. Such strengthening effect is consistent with the intramolecular bond breakage being harder to attain, which may explain why solvent evaporation does not produce phase pure DEX−CAT cocrystal.

TABLE 4
Key features in the FTIR spectra of DEX, the polyphenolic
coformers, and each DEX cocrystal systems.

| | O—H | C═O | C═C |
|---|---|---|---|
| Sample | stretching/cm⁻¹ | stretching/cm⁻¹ | stretching/cm⁻¹ |
| DEX | 3472 | 1662 | 1618, 1603 |
| CAT | 3451, 3329 | — | 1599 |
| RES | 3261 | — | 1609 |
| DEX-CAT | 3555, 3466, 3201 | 1662 | 1616, 1603 |
| DEX-RES | 3555, 3470, 3260 | 1662 | 1618, 1603 |

Because of its potential use in different therapeutic areas, DEX has been formulated in a wide range of dosage forms. Apart from the most common oral and topical routes of administration, development of intranasal DEX dry powders has garnered increasing attention for treatment of allergic rhinitis and neuroinflammation induced by Covid-19 through nose-to-brain delivery in order to achieve a fast onset of action and reduced off-target adverse effects. However, rapid drug elimination by mucociliary clearance and low fluid volume available for dissolution present major challenges for the delivery of this poorly water-soluble drug to the human nasal cavity. Intranasally administered DEX sodium phosphate shows good in vivo biodistribution and fast onset of action compared to intravenous administration.

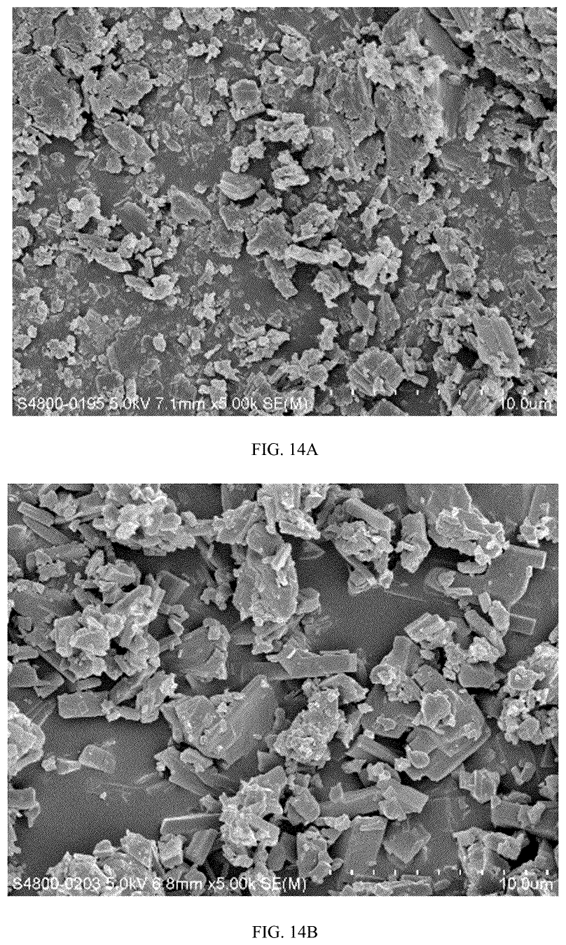

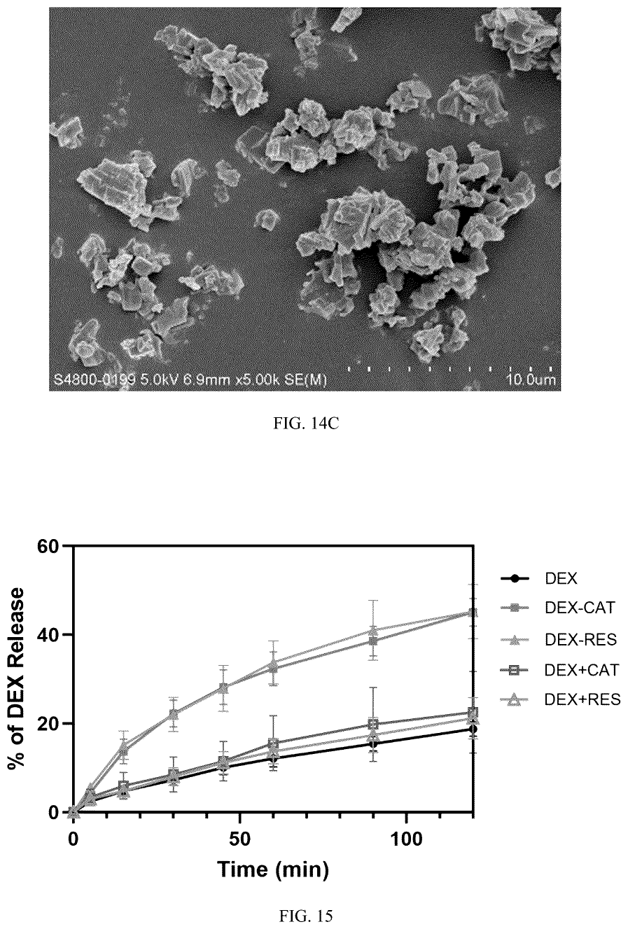

The dissolution performance of DEX−CAT and DEX−RES obtained from neat grinding in pH 5.5 simulated nasal fluid (SNF) compares favorably with raw DEX and physical mixture of DEX and respective benzenediols (DEX+CAT, DEX+RES), according to an established dissolution protocol for intranasal dry powder formulation. Micronized powders allow for adequate nasal deposition only if the particle size falls in the range 10 to 45 μm. To ensure successful deposition within the nasal cavities and to minimize the particle size effect on the initial surface-specific dissolution rates, only fraction of raw DEX and DEX cocrystals sifted to provide particle size below 63 μm (FIGS. 14A through 14C). At 120 min, the release of DEX−CAT and DEX−RES cocrystals in SNF are 2.4-fold faster than raw DEX, as indicated in FIG. 15 where DEX vs. DEX−CAT has a p value=0.006 and DEX vs. DEX−RES has a p value=0.004. The two cocrystals have a 3-fold faster dissolution than raw DEX (DEX vs. DEX−CAT p value=0.085, DEX vs. DEX−RES p value=0.051), suggesting these cocrystals have the capacity for improved DEX bioavailability with intranasal delivery. The intrinsic dissolution rate (IDR) is determined by the following equation: IDR=(dm/dt)ₘₐₓ/A, where (dm/dt)ₘₐₓ is the slope of the initial linear region of the cumulative dissolution curve until 10% of drug is dissolved, and A is the specific surface area of the dissolution sample. The volumetric size distributions of the sieved samples, which exhibit similar particle morphology, were evaluated by laser diffractometry. The D₅₀ varied from 1.52 to 24.87 μm across all formulations (Table 5). The IDR ratio of DEX cocrystals to DEX can thus be estimated by the ratio of the slope with sphere assumption, which is 76.86 (DEX−CAT) and 48.29 (DEX−RES), suggesting significant faster rates of dissolution of the DEX cocrystals than raw DEX in SNF.

TABLE 5
The volumetric size distribution of DEX, and each DEX cocrystal
system measured by laser diffraction (n = 3).

| | Volumetric size (μm) | | | |
|---|---|---|---|---|
| Formulations | D₁₀ | D₅₀ | D₉₀ | Span |
| DEX | 0.93 ± 0.16 | 1.52 ± 0.50 | 2.76 ± 1.54 | 3.15 ± 0.46 |
| DEX-CAT | 8.49 ± 1.76 | 24.87 ± 6.75 | 49.27 ± 6.42 | 5.14 ± 0.28 |
| DEX-RES | 4.46 ± 1.93 | 12.0 ± 0.42 | 35.03 ± 8.92 | 7.69 ± 0.93 |

*DEX-CAT and DEX-RES were produced by neat grinding.

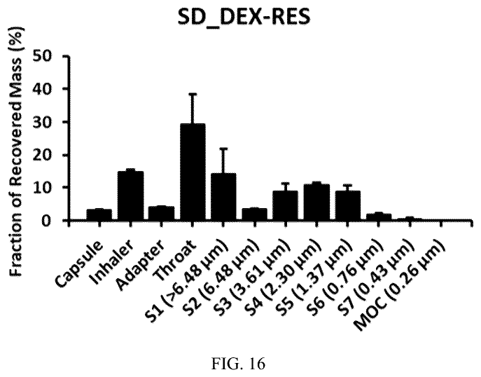

Apart from intranasal administration, development of DEX cocrystal dry powder inhalation formulation for targeted pulmonary delivery may also be beneficial in attenuating relapse and established chronic lung diseases, such as allergic asthma and chronic obstructive pulmonary disease. Particles with an aerodynamic diameter between 1 and 5 μm are considered as effective for deep lung delivery (Malcolmson and Embleton, 1998). Commercially available excipient-based DPIs are reported to produce FPFs between 10 and 50% at different flow rates (Demoly et al., 2014). In an embodiment, a DEX cocrystal dry powder inhaler (DPI) for pulmonary delivery can be formed by spray drying, which is a well-established particle engineering technique used in the pharmaceutical industry. The DEX cocrystal DPI comprises DEX and a coformer RES in 1:1 molar ratio without the aid of excipients for developing inhalable formulations. The coformer is a pharmaceutically approved antiseptic and disinfectant agent for treating various skin disorders and infections. NGI data reveals that the spray dried cocrystal formulation produced under the optimized processing parameters exhibited satisfactory aerosol performance at an inspiratory flow rate of 90 L/min, with a MMAD of 2.96±0.49 μm and a FPF of 35.43±4.97% w/w, which is comparable to that of commercial products, as indicated in Table 6, below. The NGI dispersion data for the spray dried cocrystal formulation is given in FIG. 16, which indicates that 30.26% of aerosolized powders deposited on stages 3-6, where the aerodynamic diameters fell within 3.61 to 0.76 μm.

TABLE 6
Aerodynamic size distribution (MMAD, GSD, and FPF) of spray-dried DEX-
RES formulation in 1:1 molar ratio under the selected processing parameters.
Methanol was used as the model solvent.

| | Total | | Feed | | | | |
|---|---|---|---|---|---|---|---|
| | solute | Inlet | pump | Atomizing | | | |
| | conc. | temperature | rate | air flow | MMAD | GSD | FPF |
| | (mg/mL) | (° C.) | (mL/min) | (L/h) | (μm) | (μm) | (% w/w) |
| DEX- | 1.5 | 65 | 1.5 | 742 | 2.96 ± | 2.30 ± | 35.43 ± |
| RES | | | | | 0.49 | 0.13 | 4.97 |

### MATERIALS AND METHODS

### Methodology

### Materials

DEX (≥99%) was obtained from Yick Vic Chemicals & Pharmaceuticals Limited (Hong Kong, China). The benzenediol and benzenetriol coformers: catechol (CAT), resorcinol (RES), hydroquinone (HYQ), hydroxyquinol (HXQ), phloroglucinol (PHL), and pyrogallol (PYR) were purchased from Sigma-Aldrich (St. Louis, MO, USA) and Alfa Aesar (Ward Hill, MA, USA). Potassium bromide (KBr) for FTIR analysis was sourced from J&K Scientific Limited, China. Sodium chloride (NaCl), potassium chloride (KCl), and calcium chloride (CaCl₂) for preparation of simulated nasal fluid were obtained from VWR BDH Chemicals (VWR International S.A.S., France). Ethanol, and methanol of analytical grade were obtained from VWR BDH Chemicals (VWR International S.A.S., France) and Merck KGaA (Darmstadt, Germany). Water was purified through a Thermolyne NANOpure Diamond Analytical ultra-pure water system (Barnstead, Thermo Fisher Scientific, Waltham, MA, USA).

### Preparation of DEX Cocrystals for Nasal Inhalation

Cocrystallizing of DEX with benzenediol (CAT, RES, and HYQ) and benzenetriol coformers (HXQ, PHL, and PYG) were examined using neat grinding, solution evaporation, and melt crystallization. For neat grinding, equimolar amounts (0.597 mmol) of DEX (234.3 mg) and benzendiol (65.7 mg) were mixed and ground with a mortar and pestle for approximately 15 min at ambient temperature. The powders were frequently scraped out from the mortar and pestle, and re-mixed throughout the grinding process. Prior to solid-state characterizations, the variation of particle size was minimized by passing the samples through a standard testing sieve with a diameter of 63 μm (VWR International, New York, USA). For solution evaporation, equimolar amounts (0.597 mmol) of DEX (234.3 mg) and the coformer (65.7 mg) were dissolved in a beaker with 100 mL ethanol, followed by sonication until a homogeneous solution was obtained. The solutions were sealed with pierced parafilm to allow slow evaporation in a fume hood for 72 h. Rapid solvent removal was performed using a rotary evaporator (Buchi, Germany) under a vacuum with the rotary flask being immersed in a water bath at 60° C. with a rotating speed of 60 rpm. The product was dried in an oven at 60° C. for 3 h to remove residual solvent and gently triturated to a fine powder for further analysis. For melt crystallization, a physical mixture of DEX and coformer in 1:1 molar ratio was heated at 10° C./min until a melt was formed using a differential scanning calorimeter. The molten mixture was then cooled to designated annealed temperatures at a cooling rate of 10° C./min. The mixture was kept at the annealed temperature until the crystallization process was completed, up to 24 h. All the resulting products were stored in sealed containers.

### Preparation of Spray-Dried DEX Cocrystal Powders for Oral Inhalation

The inhalable DEX cocrystal formulation for pulmonary delivery includes a solution containing equimolar amounts (0.597 mmol) of DEX (234.3 mg) and benzenediol (65.7 mg) in methanol that is spray-dried using a Büchi B-290 spray dryer with a standard two-fluid nozzle, glass chamber and a high-performance cyclone for collection of small particles (Büchi Labortechnik, Flawil, Switzerland). The spray dryer is equipped with a Buchi B-296 Dehumidifier and B-295 Inert Loop and a spray nozzle tip diameter of 0.7 mm with nitrogen as the atomization gas. The formulations were prepared according to the processing parameters listed in Table 6, above, with a rate of aspirations fixed at 100% (approximately 35 m³/h), resulting in an outlet temperature of about 44° C.

### Powder X-Ray Diffraction (PXRD) and Crystal Structure Determination

The polycrystalline cocrystals of DEX were characterized by X-ray diffraction. The measurements were done on a Rigaku SmartLab 9 kW diffractometer with a copper rotating anode (K alpha1 1.54059 Å, K alpha2 1.54441 Å) rated at 200 mA/45 kV at room temperature with a step size of 0.02 degree two-theta. Bragg Brentano CBO incident X-ray optics was used, with a 5.0 deg incident parallel Soller slit, a ½ -degree incident slit, a 1.0×10.0 mm length limiting slit, a 5.0 deg receiving parallel slit, a ½ -degree first receiving slit and a 0.3 mm second receiving slit. Diffraction signals were filtered with a K beta nickel filter, and diffraction data were collected with a HyPix-3000 detector in 1D mode.

Possible unit cell parameters were obtained by N-TREOR09 based on the diffraction patterns. Unit cells with reasonable volumes were used for further analysis. Space group determination was done by detecting the extinction group. Literature three-dimensional atomic coordinates of the individual components, CAT (Brown, C., Acta Crystallographica 1966, 21(1), 170-4), RES (Bacon, G. et al., Zeitschrift für Kristallographie-Crystalline Materials 1973, 138 (1-6), 19-40), and DEX (Raynor et al., Acta Crystallogr. Sect. Sect. E: Struct. Rep. Online 2007, 63 (6), o2791-3) were used as the initial models for simulated annealing procedures in the EXPO2014 program suite. Ten simulated annealing with ten structure solutions generated in each annealing were made for a consistent converging structure model. The structures with the lowest cost function were used for Rietveld refinement. All non-hydrogen atoms were refined isotropically. Geometrical restraints were applied on the DEX, CAT, RES molecules according to the reported crystal structures of the individual compounds, but not on the alpha-hydroxy ketone group in DEX. Hydrogen atoms were included in the idealized positions. Thermal Analysis

Differential scanning calorimetry (DSC) and thermogravimetric analysis (TGA) profiles were generated by a TA DSC 250 differential scanning calorimeter (TA Instruments, New Castle, DE, USA) and a TGA Q5000 thermogravimetric analyzer (TA Company, New Castle, DE, USA), respectively. For DSC experiments, pure indium was used for routine calibration of enthalpy and cell constant. An accurately weighed sample (˜3 mg) was encased in a Tzero Aluminum Hermetic pan (TA Instruments, New Castle, DE, USA) with pinhole vented lid if required and heated from 50° C. to 300° C. at a scanning rate of 10° C./min. In the TGA experiments, each sample (5-7 mg) was placed on an open pan and heated at 10° C./min from 50° C. to 300° C. Nitrogen was used as the purge gas at 20 mL/min for both the DSC and TGA analyses. The TA Trios Software was used for data analysis.

### Fourier-Transform Infrared Spectroscopy (FTIR)

The FTIR spectra were obtained with a FTIR spectrophotometer (Spectrum Two, PerkinElmer Instrument, USA) in a KBr diffuse reflectance mode. The scan was performed in the range of 4000 cm⁻¹ to 400 cm⁻¹ at an interval 0.5 cm⁻¹. A total of 32 scans were collected at a resolution of 4 cm⁻¹ for each sample.

### Scanning Electron Microscopy (SEM)

The particle morphology of the samples was observed by field emission scanning electron microscopy (Hitachi S-4800 FEG, Hitachi, Tokyo, Japan). The powders were sprinkled onto carbon adhesive tape mounted on SEM stubs. Any sample not adhering to the tape was removed by compressed air. A sputter coater (Bal-tec SCD 005 Sputter Coater, Bal-Tec GmbH, Schalksmühle, Germany) was used to coat the powder with approximately 11 nm gold-palladium alloy in two cycles (60 s each) to create a conductive layer and avoid overheating.

### High Performance Liquid Chromatography (HPLC)

The concentrations of DEX were quantified by HPLC equipped with a diode array detector (Agilent 1200 series, Agilent Technologies, USA) and an Agilent Zorbax Eclipse Plus C18 column (5 μm, 250 mm×4.6 mm) in an isocratic condition at 239 nm. The mobile phase consisted of a mixture of methanol and water (65:35, v/v). A 30 μL aliquot of each sample solution was injected onto the column with a flow rate of 1 mL/min. The retention time of DEX was found at 8.3 min.

### Particle Size Distribution Measurement by Laser Diffraction

The particle size and size distribution of the powders was determined using laser diffraction equipment, Mastersizer 3000 (Malvern Instruments Ltd, Worcestershire, UK) with Aero S dry powder disperser. Prior to the analysis, both raw DEX and DEX cocrystals powders were sifted with a diameter under 63 μm to control the particle size variation. The particle size distribution was calculated from the light scattering pattern using Mie theory. Particle size at 10% (D₁₀), 50% (D₅₀), 90% (D₉₀) of the volume distribution were calculated automatically using the Mastersizer 3000 software based on Fraunhofer theory. Span was calculated as (D₉₀-D₁₀)/D₅₀. All the samples were measured in triplicate.

### In-Vitro Drug Release Study for Nasal Delivery

Raw DEX powders (1.5 mg) and equimolar amount of sieved DEX cocrystal powders were separately poured into a jacketed beaker containing 50 mL of pH 5.5 simulated nasal fluid (8.77 g NaCl, 2.98 g KCl, 0.59 g CaCl₂ and distilled water up to 1000 ml), for a period of 240 min at 37±0.5° C. under sink condition. The solution was stirred at 50 rpm on a magnetic stirrer. The dissolution medium and temperature were selected to mimic the physiological condition in the nasal cavity^{35, 36}. At designated times of 5, 10, 15, 20, 30, 45, 60, 90, and 120 min, 1 mL of the dissolution medium was withdrawn and replaced with an equal volume of fresh medium. The sample solution was filtered through 0.45 μm nylon syringe filters and assayed for drug content by HPLC. The intrinsic dissolution rate (IDR) is determined by the following equation: IDR=(dm/dt)ₘₐₓ/A, where (dm/dt)ₘₐₓ is the slope of the initial linear region of the cumulative dissolution curve until 10% of drug is dissolved, and A is the specific surface area of the dissolution sample. The following assumptions were made: (i) spherical particles, (ii) constant particle size, and (iii) constant number of particles during the initial phase of the dissolution experiment under sink condition. With the assumptions, the particle size distribution data collected by laser diffractometry is used for the particle surface area (SA_{particle}) calculation. The total number of particles (n) subject to dissolution is calculated by V_{bulk}/V_{particle}, where V_{particle} is the volume of each primary particle, and V_{bulk} is the volume of compound added to the dissolution medium. The total surface area of all particles added to the dissolution medium is thus calculated through nSA_{particle}. Finally, the specific surface area (A, m²/g) defined as the total surface area of a material per unit of mass could be obtained.

### In-vitro Aerosol Performance Evaluation for Pulmonary Delivery

Assessment of the in vitro aerosol performance of the spray-dried DEX−RES powder formulation was performed using a Next Generation Impactor (NGI, Copley, Nottingham, UK). A thin layer of silicon grease (Slipicone; DC Products, Waverley, VIC, Australia) coated onto the impactor stages minimizes particle bounce. Approximately 3 mg of spray-dried DEX−RES cocrystal powders were loaded into a size 3 hydroxypropyl methylcellulose capsule (Capsugel, West Ryde, NSW, Australia), which were aerosolized by Breezhaler® (Novartis Pharmaceuticals, Hong Kong, China) at a flow rate of 90 L/min for 2.7 s. Precise amounts of methanol were used for rinsing DEX and RES in all stages. The solutions were subsequently filtered using a 0.45 μm nylon syringe filters and the solutions assayed by HPLC. The recovered dose, fine particle fraction (FPF), mass median aerodynamic diameter (MMAD), and geometric standard deviation (GSD) were calculated. The FPF is the mass fraction of the particles <5 μm with respect to the recovered dose. The recovered dose was defined as the sum of powder mass assayed on all the parts.

### Stability Study

Raw DEX and DEX cocrystal powders were stored at 25° C./75% RH for 1 month. The samples before and after the storage were collected for PXRD analysis. Drug assay before and after storage of the DEX−benzenediol cocrystal systems under 25° C./75% RH in day 0, 7, and 30 were quantified by HPLC.

### Statistical Analysis

A two-sample t-test was employed for data analysis. A p-value less than 0.05 was considered as statistically significant.

All publications referred to or cited herein are incorporated by reference in their entirety, including all figures and tables, to the extent they are not inconsistent with the explicit teachings of this specification. It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Wong et al., S. N.; Chen, Y. C. S.; Xuan, B.; Sun, C. C.; Chow, S. F., Cocrystal Engineering of Pharmaceutical Solids: Therapeutic Potentials and Challenges. CrystEngComm 2021.
Sun, C. C., Cocrystallization for successful drug delivery. Expert opinion on drug delivery 2013, 10 (2), 201-213.
Chow, S. F.; Shi, L.; Ng, W. W.; Leung, K. H. Y.; Nagapudi, K.; Sun, C. C.; Chow, A. H., Kinetic entrapment of a hidden curcumin cocrystal with phloroglucinol. Crystal growth & design 2014, 14 (10), 5079-5089.
Hasa, D.; Jones, W., Screening for new pharmaceutical solid forms using mechanochemistry: A practical guide. Advanced drug delivery reviews 2017, 117, 147-161.
Loya, J. D.; Li, S. J.; Unruh, D. K.; Hutchins, K. M., Mechanochemistry as a Tool for Crystallizing Inaccessible Solids from Viscous Liquid Components. Crystal Growth & Design 2021.
Trask, A. V.; van de Streek, J.; Motherwell, W. S.; Jones, W., Achieving polymorphic and stoichiometric diversity in cocrystal formation: Importance of solid-state grinding, powder X-ray structure determination, and seeding. Crystal growth & design 2005, 5 (6), 2233-2241.
am Ende, D. J.; Anderson, S. R.; Salan, J. S., Development and scale-up of cocrystals using resonant acoustic mixing. Organic Process Research & Development 2014, 18 (2), 331-341.
Boldyreva, E., Mechanochemistry of inorganic and organic systems: what is similar, what is different? Chem. Soc. Rev. 2013, 42 (18), 7719-7738.
FrišČiĆ, T.; Jones, W., Application of mechanochemistry in the synthesis and discovery of new pharmaceutical forms: co-crystals, salts and coordination compounds. In Pharmaceutical Salts and Co-crystals, 2011; pp 154-187.
Friscic, T.; Jones, W., Recent advances in understanding the mechanism of cocrystal formation via grinding. Cryst. Growth Des. 2009, 9 (3), 1621-1637.
Lien Nguyen, K.; Friščić, T.; Day, G. M.; Gladden, L. F.; Jones, W., Terahertz time-domain spectroscopy and the quantitative monitoring of mechanochemical cocrystal formation. Nature Materials 2007, 6 (3), 206-209.
Jayasankar, A.; Somwangthanaroj, A.; Shao, Z. J.; Rodríguez-Hornedo, N., Cocrystal formation during cogrinding and storage is mediated by amorphous phase. Pharm. Res. 2006, 23 (10), 2381-2392.
Willart, J.; Descamps, M., Solid state amorphization of pharmaceuticals. Molecular Pharmaceutics 2008, 5 (6), 905-920.
Descamps, M.; Willart, J., Perspectives on the amorphisation/milling relationship in pharmaceutical materials. Advanced Drug Delivery Reviews 2016, 100, 51-66.
MERCK & CO., I. DECADRON® package insert. https://www.accessdata.fda.gov/drugsatfda_docs/label/2004/11664slr062_decadron_lbl.pdf.
Ledford, H., Coronavirus breakthrough: dexamethasone is first drug shown to save lives. Nature 2020, 582 (7813), 469-470.
Group, T. R. C., Dexamethasone in hospitalized patients with Covid-19—preliminary report. The New England journal of medicine 2020.
Corticosteroids for COVID-19: living guidance, 2 Sep. 2020; World Health Organization: 2020.
National Center for Biotechnology Information. PubChem Compound Database. CID 5743. https://pubchem.ncbi.nlm.nih.gov/compound/5743.
Allen, F. H.; Motherwell, W. D. S.; Raithby, P. R.; Shields, G. P.; Taylor, R., Systematic analysis of the probabilities of formation of bimolecular hydrogen-bonded ring motifs in organic crystal structures. New J. Chem. 1999, 23 (1), 25-34.
Desiraju, G. R., Supramolecular synthons in crystal engineering—a new organic synthesis. Angewandte Chemie International Edition in English 1995, 34 (21), 2311-2327.
Wang, J.-R.; Yu, Q.; Dai, W.; Mei, X., Drug—drug co-crystallization presents a new opportunity for the development of stable vitamins. Chem. Commun. 2016, 52 (17), 3572-3575.
Cambridge Crystallographic Data Center. Cambridge, U.K. www.ccdc.cam.ac.uk.
Goud, N. R.; Babu, N. J.; Nangia, A., Sulfonamide-Pyridine-N-oxide Cocrystals. Crystal growth & design 2011, 11 (5), 1930-1939.
Wong, S. N.; Hu, S.; Ng, W. W.; Xu, X.; Lai, K. L.; Lee, W. Y. T.; Chow, A. H. L.; Sun, C. C.; Chow, S. F., Cocrystallization of curcumin with benzenediols and benzenetriols via rapid solvent removal. Crystal Growth & Design 2018, 18 (9), 5534-5546.
Durairaj, R. B., Resorcinol: chemistry, technology and applications. Springer Science & Business Media: 2005.
Altomare, A.; Giacovazzo, C.; Guagliardi, A.; Moliterni, A. G.; Rizzi, R.; Werner, P.-E., New techniques for indexing: N-TREOR in EXPO. J. Appl. Crystallogr. 2000, 33 (4), 1180-1186.
Brown, C., The crystal structure of catechol. Acta Crystallographica 1966, 21 (1), 170-174.
Bacon, G.; Jude, R., Neutron-diffraction studies of salicylic acid and α resorcinol. Zeitschrift für Kristallographie-Crystalline Materials 1973, 138 (1-6), 19-40.
Raynor, J. W.; Minor, W.; Chruszcz, M., Dexamethasone at 119 K. Acta Crystallogr. Sect. Sect. E: Struct. Rep. Online 2007, 63 (6), o2791-o2793.
A. Altomare, C. C., C. Giacovazzo, A. Moliterni, R. Rizzi, N. Corriero and A. Falcicchio, EXPO2014. J. Appl. Cryst. 2013, (46), 1231-1235.
Kürti, L.; Gáspár, R.; Márki, Á.; Kápolna, E.; Bocsik, A.; Veszelka, S.; Bartos, C.; Ambrus, R.; Vastag, M.; Deli, M. A., In vitro and in vivo characterization of meloxicam nanoparticles designed for nasal administration. European Journal of Pharmaceutical Sciences 2013, 50 (1), 86-92.
Mohamad, S. A.; Badawi, A. M.; Mansour, H. F., Insulin fast-dissolving film for intranasal delivery via olfactory region, a promising approach for the treatment of anosmia in COVID -19 patients: Design, in-vitro characterization and clinical evaluation. Int. J. Pharm. 2021, 601, 120600.
Bartos, C.; Pallagi, E.; Szabó-Révész, P.; Ambrus, R.; Katona, G.; Kiss, T.; Rahimi, M.; Csóka, I., Formulation of levodopa containing dry powder for nasal delivery applying the quality-by-design approach. European Journal of Pharmaceutical Sciences 2018, 123, 475-483.
Jug, M.; Hafner, A.; Lovrić, J.; Kregar, M. L.; Pepić, I.; Vanić, Ž.; Cetina-Čižmek, B.; Filipović-Grčić, J., An overview of in vitro dissolution/release methods for novel mucosal drug delivery systems. J. Pharm. Biomed. Anal. 2018, 147, 350-366.
Jug, M.; Hafner, A.; Lovrić, J.; Lusina Kregar, M.; Pepić, I.; Vanić, Ž.; Cetina-Čižmek, B.; Filipović-Grčić, J., In vitro dissolution/release methods for mucosal delivery systems. ADMET and DMPK 2017, 5 (3), 173-182.
Nayak, A.; Pedireddi, V., Rational analysis of melting point behavior of co-crystals of 4-nitrophenol with some aza-compounds. Crystal Growth & Design 2016, 16 (10), 5966-5975.
Berman, P.; Meiri, N.; Colnago, L. A.; Moraes, T. B.; Linder, C.; Levi, 0.; Parmet, Y.; Saunders, M.; Wiesman, Z., Study of liquid-phase molecular packing interactions and morphology of fatty acid methyl esters (biodiesel). Biotechnology for Biofuels 2015, 8 (1), 1-16.
Boese, R.; Kirchner, M. T.; Dunitz, J. D.; Filippini, G.; Gavezzotti, A., Solid-State Behaviour of the Dichlorobenzenes: Actual, Semi-Virtual and Virtual Crystallography. Helv. Chim. Acta 2001, 84 (6), 1561-1577.
Wang, F.-Y.; Zhang, Q.; Zhang, Z.; Gong, X.; Wang, J.-R.; Mei, X., Solid-state characterization and solubility enhancement of apremilast drug—drug cocrystals. CrystEngComm 2018, 20 (39), 5945-5948.
Weng, J.; Wong, S. N.; Xu, X.; Xuan, B.; Wang, C.; Chen, R.; Sun, C. C.; Lakerveld, R.; Kwok, P. C. L.; Chow, S. F., Cocrystal engineering of itraconazole with suberic acid via rotary evaporation and spray drying. Crystal Growth & Design 2019, 19 (5), 2736-2745.
Xuan, B.; Wong, S. N.; Zhang, Y.; Weng, J.; Tong, H. H.; Wang, C.; Sun, C. C.; Chow, S. F., Extended release of highly water-soluble isoniazid attained through cocrystallization with curcumin. Crystal Growth & Design 2020, 20 (3), 1951-1960.
Alhalaweh, A.; Alzghoul, A.; Kaialy, W.; Mahlin, D.; Bergström, C. A., Computational predictions of glass-forming ability and crystallization tendency of drug molecules. Molecular pharmaceutics 2014, 11 (9), 3123-3132.
Baird, J. A.; Van Eerdenbrugh, B.; Taylor, L. S., A classification system to assess the crystallization tendency of organic molecules from undercooled melts. J. Pharm. Sci. 2010, 99 (9), 3787-3806.
Mahlin, D.; Bergström, C. A., Early drug development predictions of glass-forming ability and physical stability of drugs. European Journal of Pharmaceutical Sciences 2013, 49 (2), 323-332.
Chattoraj, S.; Bhugra, C.; Telang, C.; Zhong, L.; Wang, Z.; Sun, C. C., Origin of two modes of non-isothermal crystallization of glasses produced by milling. Pharm. Res. 2012, 29 (4), 1020-1032.
Mithu, M. S. H.; Ross, S. A.; Hurt, A. P.; Douroumis, D., Effect of mechanochemical grinding conditions on the formation of pharmaceutical cocrystals and co-amorphous solid forms of ketoconazole-Dicarboxylic acid. Journal of Drug Delivery Science and Technology 2021, 63, 102508.
Hu, Y.; Gniado, K.; Erxleben, A.; McArdle, P., Mechanochemical reaction of sulfathiazole with carboxylic acids: Formation of a cocrystal, a salt, and coamorphous solids. Crystal growth & design 2014, 14 (2), 803-813.
Haneef, J.; Chadha, R., Drug-drug multicomponent solid forms: cocrystal, coamorphous and eutectic of three poorly soluble antihypertensive drugs using mechanochemical approach. AAPS PharmSciTech 2017, 18 (6), 2279-2290.
Aljohani, M.; MacFhionnghaile, P.; McArdle, P.; Erxleben, A., Investigation of the formation of drug-drug cocrystals and coamorphous systems of the antidiabetic drug gliclazide. Int. J. Pharm. 2019, 561, 35-42.
Jayasankar, A.; Good, D. J.; Rodríguez-Hornedo, N., Mechanisms by which moisture generates cocrystals. Molecular pharmaceutics 2007, 4 (3), 360-372.
Wong, S. N.; Chan, S. W. S.; Peng, X.; Xuan, B.; Lee, H. W.; Tong, H. H.; Chow, S. F., Effects of the Glass-Forming Ability and Annealing Conditions on Cocrystallization Behaviors via Rapid Solvent Removal: A Case Study of Voriconazole. Pharmaceutics 2020, 12 (12), 1209.
Pekar, K. B.; Lefton, J. B.; McConville, C. A.; Burleson, J.; Sethio, D.; Kraka, E.; Runcevski, T., Mechanosynthesis of a Coamorphous Formulation of Creatine with Citric Acid and Humidity-Mediated Transformation into a Cocrystal. Crystal Growth & Design 2021, 21 (2), 1297-1306.
Wu, W.; Wang, Y.; Löbmann, K.; Grohganz, H.; Rades, T., Transformations between Co-Amorphous and Co-Crystal Systems and Their Influence on the Formation and Physical Stability of Co-Amorphous Systems. Molecular pharmaceutics 2019, 16 (3), 1294-1304.
Seefeldt, K.; Miller, J.; Alvarez-Nunez, F.; Rodríguez-Hornedo, N., Crystallization pathways and kinetics of carbamazepine—nicotinamide cocrystals from the amorphous state by in situ thermomicroscopy, spectroscopy, and calorimetry studies. J. Pharm. Sci. 2007, 96 (5), 1147-1158.
Kjaergaard, H. G.; Howard, D. L.; Schofield, D. P.; Robinson, T. W.; Ishiuchi, S.-i.; Fujii, M., OH- and CH-stretching overtone spectra of catechol. The Journal of Physical Chemistry A 2002, 106 (2), 258-266.
Grieco, C.; Hanes, A. T.; Blancafort, L.; Kohler, B., Effects of intra-and intermolecular hydrogen bonding on O—H bond photodissociation pathways of a catechol derivative. The Journal of Physical Chemistry A 2019, 123 (25), 5356-5366.
Varfolomeev, M. A.; Abaidullina, D. I.; Gainutdinova, A. Z.; Solomonov, B. N., FTIR study of H-bonds cooperativity in complexes of 1, 2-dihydroxybenzene with proton acceptors in aprotic solvents: Influence of the intramolecular hydrogen bond. Spectrochimica Acta Part A: Molecular and Biomolecular Spectroscopy 2010, 77 (5), 965-972.
Nižić Nodilo, L.; Ugrina, I.; Špoljarić, D.; Amidžić Klarić, D.; Jakobušić Brala, C.; Perkušić, M.; Pepić, I.; Lovrić, J.; Saršon, V.; Safundžić Kučuk, M., A Dry Powder Platform for Nose-to-Brain Delivery of Dexamethasone: Formulation Development and Nasal Deposition Studies. Pharmaceutics 2021, 13 (6), 795.
Pérez-Osorio, I. N.; Espinosa, A.; Velázquez, M. G.; Padilla, P.; Bárcena, B.; Fragoso, G.; Jung-Cook, H.; Besedovsky, H.; Meneses, G.; Conde, E. L. S., Nose-to-brain delivery of dexamethasone: biodistribution studies in mice. J. Pharmacol. Exp. Ther. 2021, 378 (3), 244-250.
Johnson, R. M.; Vinetz, J. M., Dexamethasone in the management of covid-19. British Medical Journal Publishing Group: 2020; Vol. 370.
Schipper, N. G.; Verhoef, J.; Merkus, F. W., The nasal mucociliary clearance: relevance to nasal drug delivery. Pharm. Res. 1991, 8 (7), 807-814.
Forbes, B.; Bommer, R.; Goole, J.; Hellfritzsch, M.; De Kruijf, W.; Lambert, P.; Caivano, G.; Regard, A.; Schiaretti, F.; Trenkel, M., A consensus research agenda for optimising nasal drug delivery. Expert opinion on drug delivery 2020, 17 (2), 127-132.
Fasiolo, L. T.; Manniello, M. D.; Tratta, E.; Buttini, F.; Rossi, A.; Sonvico, F.; Bortolotti, F.; Russo, P.; Colombo, G., Opportunity and challenges of nasal powders: Drug formulation and delivery. European Journal of Pharmaceutical Sciences 2018, 113, 2-17.
Noyes, A. A.; Whitney, W. R., The rate of solution of solid substances in their own solutions. Journal of the American Chemical Society 1897, 19 (12), 930-934.
Demoly, P., Hagedoorn, P., de Boer, A. H., Frijlink, H. W., 2014. The clinical relevance of dry powder inhaler performance for drug delivery. Respiratory medicine 108, 1195-1203.
Malcolmson, R. J., Embleton, J. K., 1998. Dry powder formulations for pulmonary delivery. Pharmaceutical Science & Technology Today 1, 394-398.

We claim:

1. A cocrystal of dexamethasone (DEX), comprising DEX and at least one coformer wherein the coformer comprises a plurality of hydrogen-bonding functional groups.

2. The cocrystal according to claim 1, wherein the coformer comprises an aromatic group.

3. The cocrystal according to claim 1, wherein the coformer has a structure: 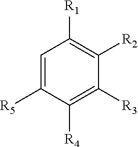
where R₁, R₂, R₃, R₄, and R₅ are independently selected from H, OH, CO₂H, NH₂, and SH, and wherein at least two of R₁, R₂, R₃, R₄, and R₅ are not H.

4. The cocrystal according to claim 1, wherein the molar ratio of DEX to the coformer is m:n where m is 1, 2, 3, or 4, and n is 0.5, 1, 2, 3, or 4.

5. The cocrystal according to claim 4, wherein m is 1 and n is 0.5, 1, or 2.

6. The cocrystal according to claim 1, wherein the coformer is selected from the group consisting of benzenediols, benzenetriols and benzenetetrols and pentahydroxybenzene.

7. The cocrystal according to claim 1, wherein the coformer is selected from the group consisting of catechol and resorcinol.

8. The cocrystal according to claim 1, wherein the cocrystal is a polycrystalline free-flow powder with a micron-scale particle size.

9. A method of preparing a cocrystal of the DEX and a coformer, comprising:
providing a mixture of DEX and at least one benzenediol; and
mechanochemical treating the mixture to form the cocrystal.

10. The method according to claim 9, wherein the mechanochemical treating comprises grinding the mixture.

11. The method according to claim 9, wherein the mixture comprises DEX and the benzenediol in a 1:1 molar ratio.

12. The method according to claim 9, wherein the benzenediol is catechol (CAT).

13. The method according to claim 9, wherein the benzenediol is resorcinol (RES).

14. The method according to claim 9, further comprising thermal annealing the DEX−benzenediol cocrystals.

15. The method according to claim 14, wherein the thermal annealing is at a temperature range of about 30° C. to about 100° C.

16. The method according to claim 14, wherein the thermal annealing is at a temperature range of about 60° C. to about 80° C.

17. The method according to claim 11, further comprising exposing the cocrystal to humidity.

18. The method according to claim 14, wherein the thermal annealing is at a humidity of about 75% relative humidity at about 25° C.

19. The method according to claim 9, further comprising sieving the cocrystal to yield particles with a size in the range of 10-45 microns.

20. A medicament comprising a cocrystal of DEX and a benzenediol in a 1:1 molar ratio of DEX to the benzenediol.

21. The medicament according to claim 20, wherein the benzenediol is catechol (CAT).

22. The medicament according to claim 20, wherein the benzenediol is resorcinol (RES).

23. The medicament according to claim 20, wherein the medicament is in a formulation for intranasal administration.

24. The medicament according to claim 20, wherein the medicament is in a formulation for inhalable pulmonary administration.

25. The medicament according to claim 20, wherein the medicament comprises at least one component for treatment of at least one of allergies, asthma, rhinitis, cancer, diabetes, anemia, ulcers, and viral infections.
